(19) United States
(12) Patent Application Publication
(10) Pub. No.: US 2016/0187331 A1
(43) Pub. Date: Jun. 30, 2016

(54) METHOD FOR ACTIVE DETECTION BIO MOLECULES

(71) Applicant: RESEARCH & BUSINESS FOUNDATION SUNGKYUNKWAN UNIVERSITY, Suwon-si (KR)

(72) Inventor: Wansoo YUN, Seongnam-si (KR)

(73) Assignee: RESEARCH & BUSINESS FOUNDATION SUNGKYUNKWAN UNIVERSITY, Suwon-si (KR)

(21) Appl. No.: 14/978,936

(22) Filed: Dec. 22, 2015

(30) Foreign Application Priority Data
Dec. 24, 2014 (KR) 10-2014-0187992

(51) Int. Cl. G01N 33/543; G01N 33/553; G01R 33/12

(52) U.S. Cl. CPC G01N 33/54333; G01R 33/1269; G01N 33/553

(57) ABSTRACT

Provided is a method of detecting biomolecules. The method of detecting biomolecules includes accommodating a plurality of metal particles to which a first molecule specifically binding to target biomolecules is attached, a plurality of magnetic particles to which a second molecule specifically binding to target biomolecules is attached, and the biomolecules; applying a magnetic field to exert an attractive force on magnetic particles; filtering one or more among metal particles, biomolecules and metal particle-binding biomolecules, on which an attractive force is not exerted; and exerting an attractive force on the metal particles of a first assembly in which both of the metal particles and the magnetic particles are bound to the biomolecules by applying an electric field and detecting the metal particles.

### CROSS-REFERENCE TO RELATED APPLICATION

This application claims the benefit under 35 USC 119(a) of Korean Patent Application No. 10-2014-0187992 filed on Dec. 24, 2014 in the Korean Intellectual Property Office, the entire disclosure of which is incorporated herein by reference for all purposes.

### BACKGROUND

1. Field of the Invention

The present invention relates to a method of detecting biomolecules, and more particularly, to a method of detecting biomolecules to actively detect target biomolecules using magnetic particles, metal particles and a nanogap electrode.

2. Discussion of Related Art

While, globally, research on detection of biomolecules tends to increase annually and has attracted the attention of many scientists, techniques of detecting biomolecules are still at an early stage. To more rapidly and exactly detect biomolecules, a method of more precisely detecting biomolecules in a biological environment based on new technology of overcoming the limitation of the current technology is needed, and to this end, biocompatible nanomaterial and a high functional nanodevice may be designed and manufactured to properly interact with biomolecules.

As conventional biosensor techniques, techniques of detecting biomolecules by optical, electrical and electrochemical methods using a nano-optical probe, a nanotube or a nanowire have been developed, but a detection rate is slow and sensitivity is decreased.

The optical method using a nano-optical probe uses a measurement method of detecting a reaction with a target material to be analyzed by labeling a light emitting material with a recognizing material through an optical signal emitted from the light emitting material. Such a biosensor can perform detection within a relatively short period, but sensitivity and reliability are relatively low.

Since the electrical method using a nanowire or nanotube is highly sensitive in the detection of biomolecules, but difficult to align nanowires or nanotubes to have a desired position and shape, the reproducibility and reliability of the device are poor. Also, it is necessary to reduce the size of the device in the process of increasing the sensitivity of the device, and in this case, since it takes much time to fix a target material to the device due to a decreased size of the device, the total time for detecting biomolecules is longer.

The conventional method of detecting biomolecules (Korean Patent Application Publication No. 10-2006-0089101) uses a field effect transistor (FET), and is a labeling method in which a small active area is capable of the detection of biomolecules and the immobilization of a probe is required, and thus takes a long time to detect biomolecules. In addition, the recently-disclosed device and method of detecting biomolecules (Korean Patent Application Publication No. 10-2014-0068188) are characterized by using an optical method. However, the conventional device of detecting biomolecules does not have a fast detection rate and has a limitation in detecting low-concentration biomolecules. Particularly, there is no detection method simultaneously having a high detection rate and high sensitivity.

This is because, when the device is made small to increase sensitivity, it takes more time to encounter a target material with the device, and when the device is made large to reduce the time to encounter the target material with the device, the sensitivity of the device is necessarily reduced. This is because it is an inactive method dependent on the diffusion of the target material, which has a problem of requiring a long time to detect biomolecules from the small device having high sensitivity.

The conventional detection technique was combined with nanotechnology, successfully leading to high selectivity and reliability. However, as described above, the inactive detection method depending on the diffusion of biomolecules remains the obstacle to simultaneous achievement of a high sensitivity and a high detection rate, which are necessary for substantial application of the technology.

### SUMMARY OF THE INVENTION

The present invention is directed to providing a method of actively detecting target biomolecules, and detecting a low concentration of biomolecules at a high detection rate.

In one aspect of the present invention, a method of detecting biomolecules according to an exemplary embodiment of the present invention may include accommodating a plurality of metal particles to which a first molecule, which specifically binds to target biomolecules is attached, a plurality of magnetic particles to which a second molecule which specifically binds to the target biomolecules is attached, and the biomolecules; exerting an attractive force on the magnetic particles by applying a magnetic field; filtering one or more among metal particles, biomolecules and metal particle-binding biomolecules, on which an attractive force is not exerted; and exerting an attractive force on the metal particles of a first assembly in which both of the metal particles and the magnetic particles are bound to the biomolecules by applying an electric field and detecting the metal particles.

In one exemplary embodiment, the exerting of an attractive force on the metal particles of the first assembly in which the metal particles, the biomolecules and the magnetic particles are bound by applying an electric field and detecting of the metal particles may include generating an electric field by providing power to a first electrode and a second electrode of a metal particle-detecting structure including the first electrode and the second electrode, which are spaced apart from each other, a first metal layer formed on a surface of the first electrode and having a fine irregular surface and a second metal layer formed on a surface of the second electrode and having a fine irregular surface; electrically connecting the first metal layer with the second metal layer when the metal particles of the first assembly are attached to one or more among the first metal layer and the second metal layer by the attractive force of the electric field or when the metal particles of the first assembly are present between the first metal layer and the second metal layer; and measuring electrical properties between the first metal layer and the second metal layer.

Since a distance between the first metal layer and the second metal layer may be a very short distance in nanometer units, even when the metal particles of the first assembly are present between the first metal layer and the second metal layer, the first metal layer and the second metal layer may be electrically connected to each other.

In another aspect, a method of detecting biomolecules of the present invention according to another exemplary embodiment of the present invention may include accommodating a plurality of metal particles to which a first molecule which specifically binds to target biomolecules is attached, a plurality of magnetic particles to which a second molecule which specifically binds to the target biomolecules is attached, and the biomolecules; exerting an attractive force on the magnetic particles by applying a magnetic field; filtering one or more among metal particles, biomolecules and the metal particle-binding biomolecules, on which an attractive force is not exerted; cleaving one or more among specific bindings between the magnetic particles and the biomolecules of the first assembly in which all of the metal particles and the magnetic particles are bound to the biomolecules and specific bindings between the metal particles and the biomolecules of the first assembly; and exerting an attractive force on the metal particles by applying an electric field and detecting the metal particles.

In one exemplary embodiment, the specific binding may be cleaved by applying ultrasonic waves.

In one exemplary embodiment, the exerting of an attractive force on the metal particles by applying an electric field and detecting of the metal particles may include generating electric field by providing power to a first electrode and a second electrode of a metal particle-detecting structure including the first electrode and the second electrode, which are spaced apart from each other, a first metal layer formed on a surface of the first electrode and having a fine irregular surface and a second metal layer formed on a surface of the second electrode and having a fine irregular surface; electrically connecting the first metal layer with the second metal layer when the metal particles are attached to one or more among the first metal layer and the second metal layer by the attractive force of the electric field or when the metal particles are present between the first metal layer and the second metal layer; and measuring electrical properties between the first metal layer and the second metal layer.

Since a distance between the first metal layer and the second metal layer may be a very short distance in nanometer units, even when the metal particles are present between the first metal layer and the second metal layer, the first metal layer and the second metal layer may be electrically connected to each other.

In one exemplary embodiment, the filtering of the metal particles, biomolecules and metal particle-binding biomolecules may include filtering metal particles, biomolecules and metal particle-binding biomolecules on which an attractive force is not exerted by providing a solution.

In one exemplary embodiment, when the magnetic particles are conductive, an insulating film formed on a surface of the magnetic particles may be further included.

In one exemplary embodiment, the first electrode may include a first body having a long rod shape; and a plurality of first protrusions obliquely projecting from one side of the first body.

In one exemplary embodiment, the second electrode may include a second body having a long rod shape; and a plurality of second protrusions obliquely projecting out from one side of the second body, and the second protrusions may be disposed in the space between the first protrusions.

In one exemplary embodiment, the electrical properties may include one or more among variations of a resistance between the first metal layer and the second metal layer, a current flowing between the first metal layer and the second metal layer, and a voltage between the first metal layer and the second metal layer.

In one exemplary embodiment, the power provided to the first electrode and the second electrode may be an alternating current power. The provided alternating current power may be, for example, an alternating current voltage having a frequency of 100 kHz and a peak-to-peak voltage of about 2 to 3V.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a flowchart illustrating a method of detecting biomolecules according to an exemplary embodiment of the present invention;

FIG. 2 is a diagram illustrating a metal particle and magnetic particle according to an exemplary embodiment of the present invention;

FIG. 3 is a diagram illustrating that a metal particle and a magnetic particle are bound to a target biomolecule to be detected according to an exemplary embodiment of the present invention;

FIG. 4 is a diagram illustrating a metal particle-detecting structure according to another exemplary embodiment of the present invention;

FIG. 5 is an SEM image of FIG. 4;

FIG. 6 is a flowchart illustrating a method of detecting biomolecules according to another exemplary embodiment of the present invention; and

FIG. 7 is an image illustrating a metal particle-detected state by a method of detecting biomolecules according to another exemplary embodiment of the present invention.

### DETAILED DESCRIPTION OF EXEMPLARY EMBODIMENTS

The present invention may be modified in various ways, and have various exemplary embodiments, and specific exemplary embodiments will be illustrated in drawings and explained in detail in the detail description. However, it should be interpreted that the present invention is not limited to specific exemplary embodiments, and includes all of modifications, equivalents and alternatives included in the spirit and technical scope of the present invention.

Hereinafter, one exemplary embodiment will be described in detail with reference to the accompanying drawings. In the drawings, like numerals denote like elements.

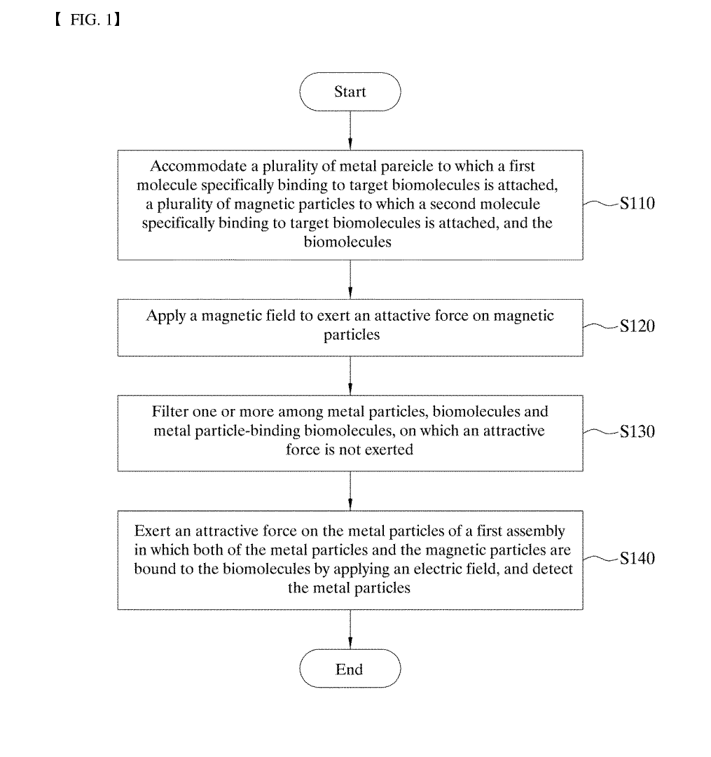

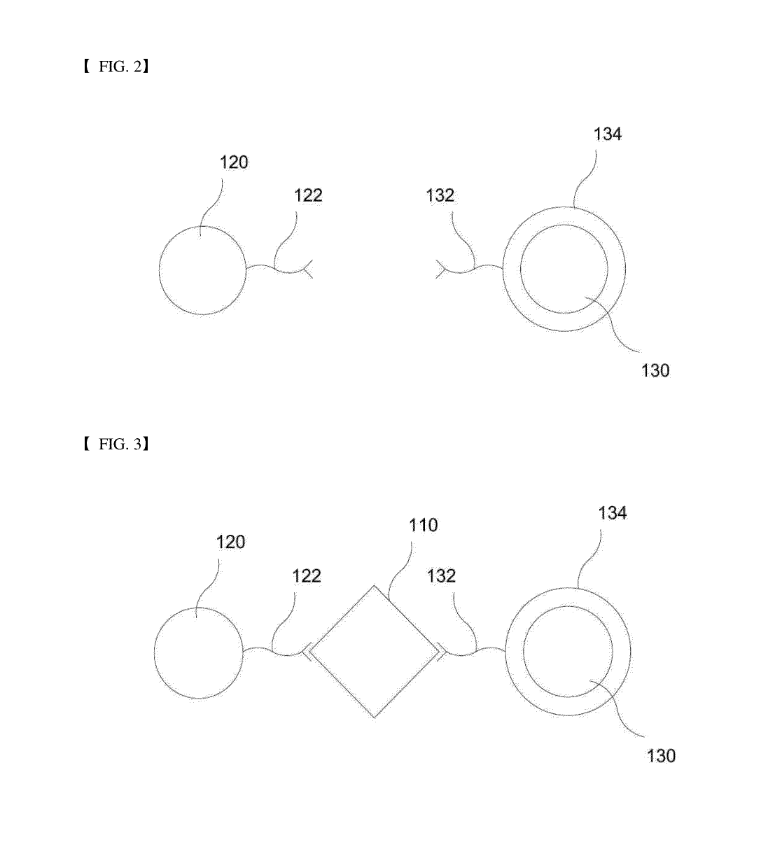

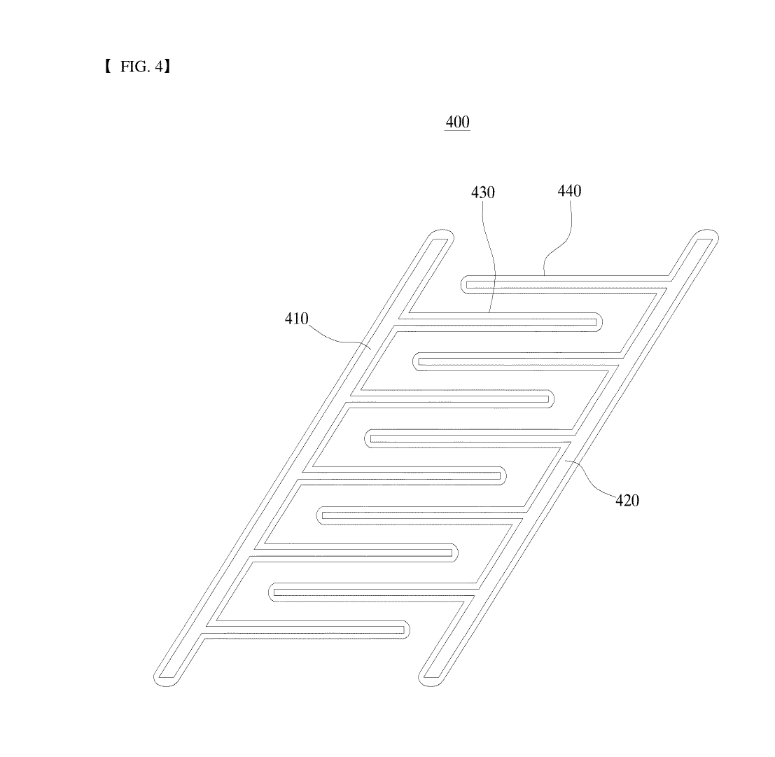

FIG. 1 is a flowchart illustrating a method of detecting biomolecules according to an exemplary embodiment of the present invention, FIG. 2 is a diagram illustrating a metal particle and magnetic particle according to an exemplary embodiment of the present invention, and FIG. 3 is a diagram illustrating that a metal particle and a magnetic particle bind to a target biomolecules according to an exemplary embodiment of the present invention.

Referring to FIGS. 1 to 3, the method of detecting biomolecules according to an exemplary embodiment of the present invention may include accommodating a plurality of metal particles to which a first molecule which specifically binds to target biomolecules is attached, a plurality of magnetic particles to which a second molecule which specifically binds to the target biomolecules is attached, and the biomolecules (S110); exerting an attractive force on the magnetic particles by applying a magnetic field (S120); filtering one or more of metal particles, biomolecules and the metal particle-binding biomolecules, on which an attractive force is not exerted (S130); and exerting an attractive force on the metal particles of a first assembly in which the metal particles, the biomolecules and the magnetic particles are bound by applying an electric field and detecting the metal particles (S140).

To actively detect biomolecules, a plurality of metal particles to which a first molecule which specifically binds to target biomolecules is attached, a plurality of magnetic particles to which a second molecule which specifically binds to the target biomolecules is attached, and the biomolecules, are accommodated (S110). In one example, a plurality of metal particles 120 to which a first molecule 122 which specifically binds to target biomolecules 110 is attached, a plurality of magnetic particles 130 to which a second molecule 132 which specifically binds to target biomolecules 110 is attached, and target biomolecules 110 are input and thus accommodated in an accommodation unit in which a liquid is accommodated, for example, a beaker.

The accommodation unit may accommodate a liquid, and when the target biomolecules 110, the plurality of metal particles 120 and the plurality of magnetic particles 130 are input into the liquid, the target biomolecules 100, the plurality of metal particles 120 and the plurality of magnetic particles 130 may freely move and react by binding to each other, thereby forming assemblies. Also, the target biomolecules 100, the plurality of metal particles 120 and the plurality of magnetic particles 130, which are input into the liquid, are mixed, and therefore a binding rate may be increased. In one example, as a liquid, deionized water may be used, but the present invention is not limited thereto.

After the biomolecules 100, the plurality of metal particles 120 and the plurality of magnetic particles 130 react with each other, target biomolecules 110, metal particles 120 and magnetic particles 130, which do not react with each other, may each be present in the accommodation unit, react with each other to have specific bindings, and therefore a first assembly in which both of the metal particles 120 and the magnetic particles 130 are bound to the target biomolecules, a second assembly in which the target biomolecules 110 and the metal particles 120 are bound, and a third assembly in which the target biomolecules 110 and the magnetic particles 130 are bound may be present in the accommodation unit.

In one example, the metal particles 120 may be metal nanoparticles, and for example, gold (Au) nanoparticles may be used as the metal particles 120, but the present invention is not limited thereto.

The metal particles 120 may be attached to the surface of a first molecule 122 specifically binding to the target biomolecules 110. In one example, the target biomolecules 110 may be target antigens, and flu, pneumonia or malaria antigens may be the target antigens, but the types of the antigens are not limited thereto.

In one example, the first molecule 122 may be a polyclonal or monoclonal antibody capable of specifically binding to the antigen.

The magnetic particles 130 may be magnetic nanoparticles, and a second molecule 132 specifically binding to the target biomolecules 110 may be attached to the magnetic particles 130.

In one example, the second molecule 132 may be a polyclonal or monoclonal antibody.

While both of the polyclonal antibody and the monoclonal antibody recognize one antigen, the polyclonal antibody is recognized by various epitopes of an antigen, and the monoclonal antibody is recognized only by one epitope of an antigen.

The magnetic particles 130 may be conductive or non-conductive, and when conductive, an insulating film 134 may be formed on a surface of the magnetic particles 130. This is because, when the magnetic particles 130 are attached to one or more of the surfaces of a first metal layer 430 and a second metal layer 440, which will be described below, or when the magnetic particles 130 are present in the space between the first metal layer 430 and the second metal layer 440, the first metal layer 430 and the second metal layer 440 are prevented from being electrically connected to each other. As the magnetic particles 120, for example, Ni nanoparticles, a Ni nanorod including nickel, or iron oxide nanoparticles (Fe₃O₄ nanoparticles, Fe₂O₃ nanoparticles) may be used, but the present invention is not limited thereto.

The target biomolecules 110 may have respective regions that specifically bind to the first molecule 122 and the second molecule 132, and the first molecule 122 and the second molecule 132 may be respectively bound to the regions. In one example, when the target biomolecules 110 are antigens, the first molecule 122 and the second molecule 132 may be specific antibodies for specifically binding to the antigens, and each of the first molecule 122 and the second molecule 132 may be specifically bound to the target biomolecules 110 through an antigen-antibody reaction. The antigen-antibody reaction is the same as the generally used method, and thus the detail description thereof will be omitted. Each of the metal particles 120 and the magnetic particles 130 may be bound to one target biomolecule to be detected 110 through such an antigen-antibody reaction.

Subsequently, an attractive force is exerted on the magnetic particles by applying a magnetic field (S120). The exertion of an attractive force on the magnetic particles 130 is to prevent filtering of the magnetic particles 130 or the assemblies binding to the magnetic particles 130. To exert an attractive force on the magnetic particles 130 present in the accommodation unit, for example, a permanent magnet or an electromagnet generating a magnetic field, or a magnetic field generator capable of generating a magnetic field may be used.

In one example, when a permanent magnet is used to apply a magnetic field, the permanent magnet is placed near the accommodation unit to apply a magnetic field to the accommodation unit, and as the accommodation unit is spaced a longer distance apart from the permanent magnet, the application of a magnetic field to the accommodation unit may be prevented.

In one example, when an electromagnet is used to apply a magnetic field, a magnetic field may or may not be applied to the accommodation unit by providing or not providing a current to the electromagnet.

In one example, when a magnetic field generator is used to apply a magnetic field, the magnetic field may or may not be applied to the accommodation unit through on/off of the magnetic field generator.

When the magnetic field is applied to the accommodation unit, an attractive force may act on the magnetic particles 130 due to the influence of the magnetic field, and the attractive force may act on one or more of the magnetic particles 130, a first assembly in which all of the metal particles 120 and the magnetic particles 130 are bound to the target biomolecules 110, and a third assembly in which the target biomolecules 110 and the magnetic particles 130 are bound in the accommodation unit.

Contrarily, an attractive force may not act on one or more of the target biomolecules 110, the metal particles 120, and a second assembly in which the target biomolecules 110 and the metal particles 120 are bound, which do not bind to the magnetic particles 130.

While an attractive force caused by a magnetic field is exerted, one or more of the metal particles, the biomolecules and the metal particle-binding biomolecules, on which the attractive force is not exerted, are filtered (S130).

One or more of the metal particles 120, the target biomolecules 110 and the second assembly in which the target biomolecules 110 and the metal particles 120 are bound, on which an attractive force caused by a magnetic field is not exerted, may be filtered. That is, materials on which the attractive force caused by a magnetic field does not act may be screened.

By providing a solution in the accommodation unit, the metal particles 120, the target biomolecules 110 and the second assembly in which the target biomolecules 110 and the metal particles 120 are bound, on which the attractive force caused by a magnetic field is not exerted, may be discharged to an outside of the accommodation unit, along with the solution provided in the accommodation unit. This is because the metal particles 120, the target biomolecules 110 and the second assembly in which the target biomolecules 110 and the metal particles 120, on which the attractive force caused by a magnetic field is not exerted, are discharged along with the solution provided in the accommodation unit to be discharged. To this end, the accommodation unit may include a first opening (not shown) capable of receiving the solution and a second opening (not shown) capable of discharging the solution.

When the filtering is completed, the magnetic particles 130, the first assembly in which all of the metal particles 120 and the magnetic particles 130 are bound to the target biomolecules 110 and the third assembly in which the target biomolecules 110 and the magnetic particles 130 are bound may be present in the accommodation unit.

Subsequently, an attractive force is exerted on the metal particles of the first assembly in which the metal particles, the biomolecules and the magnetic particles are bound by applying a magnetic field, and the metal particles are detected (S140). To this end, S140 may include generating an electric field by applying power to a first electrode 410 and a second electrode 420 of a metal particle-detecting structure 400 including the first electrode 410 and the second electrode 420, which are spaced apart from each other, the first metal layer 430 formed on a surface of the first electrode 410 and having a fine irregular surface, and the second metal layer 440 formed on a surface of the second electrode 420 and having a fine irregular surface (S142); electrically connecting the first metal layer 430 with the second metal layer 440 when the metal particles of the first assembly are attached to one or more of the first metal layer 430 and the second metal layer 440 by the attractive force of the electric field or when the metal particles of the first assembly are present in a space between the first metal layer 430 and the second metal layer 440 (S144); and measuring electrical properties between the first metal layer 430 and the second metal layer 440 (S146).

To detect the metal particles of the first assembly, an electric field is generated by applying power to the first electrode 410 and the second electrode 420 of the metal particle-detecting structure 400 including the first electrode 410 and the second electrode 420 which are spaced apart from each other, the first metal layer 430 formed on a surface of the first electrode 410 and having a fine irregular surface, and the second metal layer 440 formed on a surface of the second electrode 420 and having a fine irregular surface (S142). In one example, the metal particle-detecting structure 400 may be disposed in the accommodation unit.

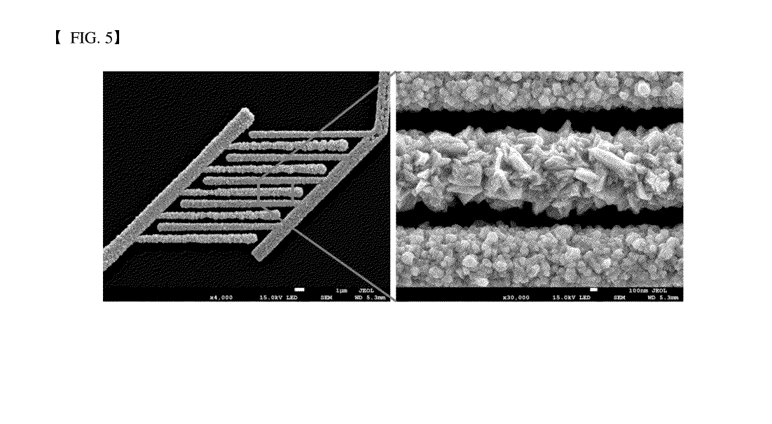

FIG. 4 is a diagram illustrating a metal particle-detecting structure according to another exemplary embodiment of the present invention, and FIG. 5 is an SEM image of FIG. 4.

Referring to FIGS. 4 and 5, the metal particle-detecting structure may include a first electrode 410, a second electrode 420, a first metal layer 430 and a second metal layer 440.

The first electrode 410 may include a first body having a long rod shape and a plurality of first protrusions obliquely projecting from one side of the first body.

The second electrode 420 may include a second body having a long rod shape and a plurality of second protrusions obliquely projecting from one side of the second body, and the second projections may be disposed between the first projections.

The first metal layer 430 may be formed on a surface of the first electrode 410, and have a fine irregular surface. The first metal layer 430 may be formed through a plating process.

The second metal layer 440 may be formed on a surface of the second electrode 420, and have a fine irregular surface. The second metal layer 440 may be formed by a plating process.

Formation of First Metal Layer 430 and Second Metal Layer 440

The first electrode 410 and the second electrode 420 may be exposed to a plating solution by being immersed in the plating solution or dropping the plating solution thereon. In one example, as the plating solution, a hydrogen tetrachloroaurate (III)(HAuCl₄) aqueous solution may be used. For example, the first electrode 410 or the second electrode 420 may be exposed to the plating solution by dropping 3 μl of the hydrogen tetrachloroaurate (III) (HAuCl₄) aqueous solution having a concentration of 800 micromoles (μM) onto the first electrode 410 or the second electrode 420.

While the first electrode 410 or the second electrode 420 is exposed to the plating solution, the first electrode 410 is grounded, and a function generator is connected to the second electrode 420, which is the other electrode, and therefore an alternating current voltage having a frequency of 100 to 10 kHz, an offset voltage of 500 mV, and a peak-to-peak voltage of 250 mV is applied.

The function generator is connected to the grounded first electrode 410, and the second electrode 420 connected with the function generator is grounded, thereby applying the same alternating current voltage as used above, and thus the first metal layer 430 and the second metal layer 440 may be formed on the first electrode 410 and the second electrode 420, respectively.

The first metal layer 430 formed on the first electrode 410 and the second metal layer 440 formed on the second electrode 420 may be spaced apart from each other, and the distance between the layers may vary depending on the positions thereof in a range of 1 to 10,000 nm. For example, the distance may be about 1/10 to 10 times the size of the metal particles 120. The distance may depend on the size of the metal particles 120 of the first assembly, which will be attached.

When the first metal layer 430 is formed on the surface of the first electrode 410, and the second metal layer 440 is formed on the surface of the second electrode 420, the distance between the first electrode 410 and the second electrode 420 becomes smaller. When the same voltage is applied and the distance is smaller, an electric field is larger. Therefore, when a voltage is applied to the first electrode 410 and the second electrode 420, the intensity of the electric field in the space between the first metal layer 430 and the second metal layer 440 may be increased. When the intensity of the electric field is increased, the metal particles 120 of the first assembly may be more strongly drawn into the space between the first metal layer 430 and the second metal layer 440, and therefore the metal particles 120 of the first assembly may be attached to one or more of the first electrode 410 and the second electrode 420.

Since the first metal layer 430 and the second metal layer 440 have a fine irregular surface, when a voltage is applied to the first electrode 410 and the second electrode 420, a non-uniform electric field may be formed in the space between the first metal layer 430 and the second metal layer 440. When the electric field is non-uniformly formed, a high electric field may be generated in a specific space between the first electrode 410 and the second electrode 420. Therefore, a higher attractive force may be exerted on the metal particles 120, and thus there is a higher possibility of attaching the metal particles 120 of the first assembly.

Each of the heights of the irregular surfaces of the first metal layer 430 and the second metal layer 440 may be about 1 to 10 μm, and preferably, about 3 nm to 5 μm. When the height is less than about 3 nm, there is an insignificant effect of forming a non-uniform electric field due to the fine irregular surface, and when the height is about 5 μm or more, the distance between the first metal layer 430 and the second metal layer 440 is decreased, and therefore there is a higher possibility of electrically communicating the first metal layer 430 with the second metal layer 440 even when the metal particles 120 of the first assembly is not attached.

When the space between the first metal layer 430 and the second metal layer 440 is very small, the intensity of the electric field may be relatively higher than that of the larger space, and therefore, the metal particles 120 of the first assembly which may not be attached to the larger space may be more easily attached.

When power is provided to the first electrode 410 and the second electrode 420 of the metal particle-detecting structure 400, an electric field may be generated in the space between the first electrode 410 and the second electrode 420. In one example, the provided power may be alternating current power, and when the alternating current power is provided, the electric field generated in the space between the first electrode 410 and the second electrode 420 may be more non-uniformly generated. In one example, to provide power, a function generator including alternating current power and a resistor may be used.

Subsequently, when the metal particles 120 of the first assembly are attached to one or more of the first metal layer 430 and the second metal layer 440 by an attractive force of the generated electric field or when the metal particles 120 of the first assembly are present in the space between the first metal layer 430 and the second metal layer 440, the first metal layer 430 and the second metal layer 440 are electrically connected (S144).

In one example, the metal particles 120 of the first assembly may be attached to one or more of the first metal layer 430 and the second metal layer 440 by dielectrophoresis or present in the space between the first metal layer 430 and the second metal layer 440. Dielectrophoresis is the application of a force to a dielectric material using a non-uniform electric field, and when the electric field is applied to a dielectric material, a positive charge and a negative charge are separated, thereby forming an electric dipole. When the electric dipole is formed, a dielectric material is transferred to a region in which the electric field is formed by the attractive force of the electric field. Therefore, when the electric field is formed in the space between the first metal layer 430 and the second metal layer 440, the metal particles 120 of the first assembly may be attached to one or more of the first metal layer 430 and the second metal layer 440 by dielectrophoresis or present in the space between the first metal layer 430 and the second metal layer 440.

Subsequently, when the first metal layer 430 and the second metal layer 440 are electrically connected, electrical properties between the first metal layer 430 and the second metal layer 440 are measured (S146). However, when the first metal layer 430 and the second metal layer 440 are not electrically connected, and even when electrically connected by the metal particles 120 of the first assembly, one or more of the electrical properties, for example, a resistance between the first metal layer 430 and the second metal layer 440, a current flowing between the first metal layer 430 and the second metal layer 440, and a voltage applied between the first metal layer 430 and the second metal layer 440 may be changed. In one example, when power is provided to apply an electric field, the metal particles 120 of the first assembly are attached to one or more of the first electrode 410 and the second electrode 420 or the metal particles 120 of the first assembly are present in the space between the first metal layer 430 and the second metal layer 440 to electrically connect the first metal layer 430 with the second metal layer 440, and thereby the change of the electrical properties of the provided power, for example, the current or voltage, or the resistance between the first metal layer 430 and the second metal layer 440 is confirmed, and therefore a user can confirm that the target biomolecules 110 are present.

To measure the electrical properties, for example, an oscilloscope or a semiconductor parameter analyzer may be used.

As described above, in the present invention, as the attractive force is exerted on the metal particles 120 of the first assembly, the presence of the target biomolecules 110 may be actively confirmed, and the presence of the target biomolecules 110 may be more rapidly confirmed.

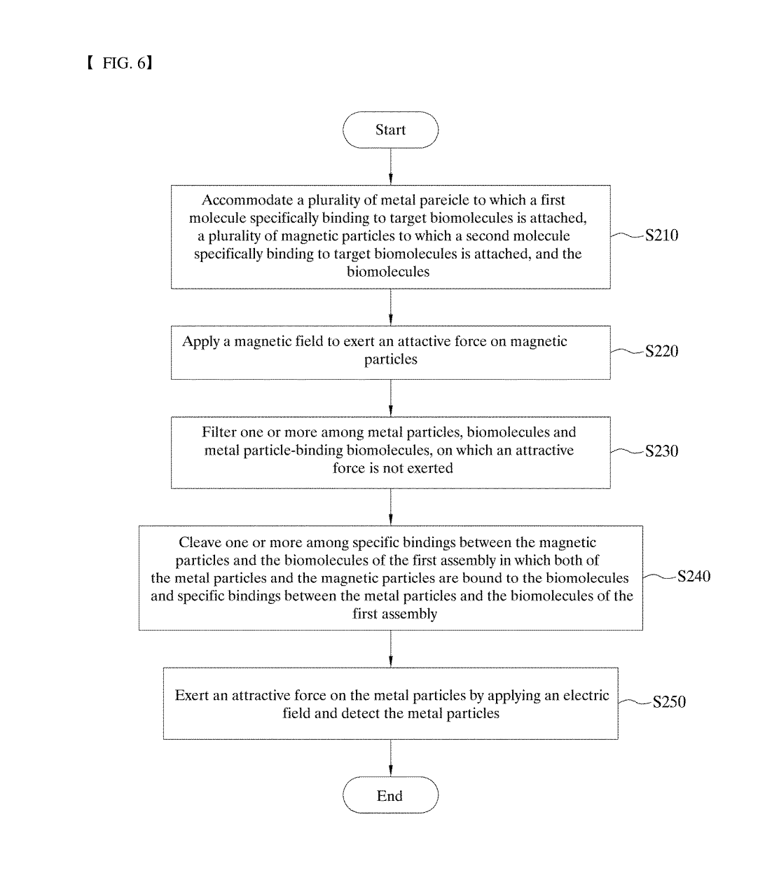

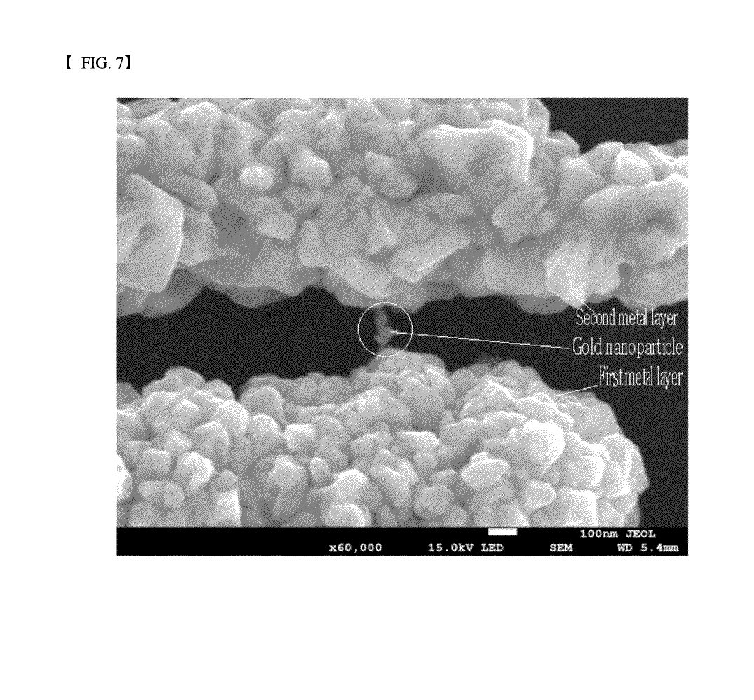

FIG. 6 is a flowchart illustrating a method of detecting biomolecules according to another exemplary embodiment of the present invention, and FIG. 7 is an image illustrating a metal particle-detected state by a method of detecting biomolecules according to another exemplary embodiment of the present invention.

Referring to FIGS. 6 and 7, the method of detecting biomolecules according to another exemplary embodiment of the present invention may include accommodating a plurality of metal particles to which a first molecule specifically binding to target biomolecules is attached, a plurality of magnetic particles to which a second molecule specifically binding to the target biomolecules is attached and the biomolecules (S210), exerting an attractive force on magnetic particles by applying a magnetic field (S220), filtering one or more of metal particles, biomolecules and metal particle-binding biomolecules, on which the attractive force is not exerted (S230), cleaving one or more of the specific bindings of the biomolecules with the magnetic particles of the first assembly in which all of the metal particles and the magnetic particles are bound to the biomolecules and the specific bindings of the biomolecules with the metal particles of the first assembly (S240), and exerting an attractive force on the metal particles by applying an electric field and detecting the metal particles (S250).

The accommodating of a plurality of metal particles to which a first molecule specifically binding to target biomolecules is attached, a plurality of magnetic particles to which a second molecule specifically binding to the target biomolecules is attached and the biomolecules (S210) is performed in the same manner as described in S110, and thus the detail description thereof will be omitted.

The exerting of an attractive force to the magnetic particles by applying a magnetic field (S220) is performed in the same manner as described in S120, and thus the detail description thereof will be omitted.

The filtering of one or more of metal particles, biomolecules and metal particle-binding biomolecules, on which the attractive force is not exerted (S230), may be performed in the same manner as described in S130, and thus the detail description thereof will be omitted.

After the filtering is completed, the magnetic particles 130, the first assembly in which all of the metal particles 120 and the magnetic particles 130 are bound to the target biomolecules 110 and the third assembly in which the target biomolecules 110 and the magnetic particles 130 are bound may be present in the accommodation unit. In this state, one or more of specific bindings between the magnetic particles 130 and the biomolecules 110 of the first assembly in which both of the metal particles 120 and the magnetic particles 130 are bound to the biomolecules 110 and specific bindings between the metal particles 120 and the biomolecules 110 of the first assembly are cleaved (S240). This is to ensure that only the metal particles 120 are detected by cleaving the specific bindings. Since the detected metal particles 120 were once specifically bound to the target biomolecules 110 and then disconnected, although only the metal particles 120 are detected, it can be confirmed that the target biomolecules 110 are present in the accommodation unit. Also, since the first assembly includes the magnetic particles 130, and the magnetic particles 130 are likely to interfere with the attractive force of the electric field, specific bindings between the target biomolecules 110 and the magnetic particles 130 of the first assembly are cleaved, the interference with the attractive force caused by the electric field acting on the metal particles 120 may be prevented.

Such specific bindings may be cleaved by applying ultrasonic waves, and for example, performed by applying ultrasonic waves while the accommodation unit is disposed in an ultrasonic cleaner. Also, in one example, the specific binding may be cleaved by heating the accommodation unit or adjusting the pH of the accommodation unit.

Subsequently, the attractive force is exerted on the metal particles 120 by applying an electric field, thereby detecting the metal particles 120 (S250). To this end, S250 may include generating an electric field by providing power to a first electrode 410 and a second electrode 420 of a metal particle-detecting structure 400 including the first electrode 410 and the second electrode 420, which are spaced apart from each other, a first metal layer 430 formed on a surface of the first electrode 410 and having a fine irregular surface and a second metal layer 440 formed on a surface of the second electrode 420 and having a fine irregular surface (S252), electrically connecting the first metal layer 430 with the second metal layer 440 when the metal particles 120 are attached to one or more of the first metal layer 430 and the second metal layer 440 by the attractive force of the electric field or when the metal particles 120 are present in the space between the first metal layer 430 and the second metal layer 440 (S254), and measuring electrical properties between the first metal layer 430 and the second metal layer 440 (S256).

To detect the metal particles 120, an electric field is generated by providing power to the first electrode 410 and the second electrode 420 of the metal particle-detecting structure 400 including the first electrode 410 and the second electrode 420, which are spaced apart from each other, the first metal layer 430 formed on a surface of the first electrode 410 and having a fine irregular surface and the second metal layer 440 formed on a surface of the second electrode 420 and having a fine irregular surface (S252).

The metal particle-detecting structure 400 used to generate the electric field is the same as described above, and the generation of the electric field using the metal particle-detecting structure 400 is performed in the same manner as described above, and thus the detail description thereof will be omitted.

When the electric field is generated, the first metal layer is electrically connected with the second metal layer when the metal particles 120 are attached to one or more of the first metal layer 430 and the second metal layer 440 by the attractive force of the electric field or when the metal particles 120 are present in the space between the first metal layer and the second metal layer (S254). That is, the first metal layer is electrically connected with the second metal layer. The attachment of the metal particles 120 may be confirmed with reference to FIG. 7. Subsequently, the electrical properties between the first metal layer 430 and the second metal layer 440 are measured (S256). S254 is performed in the same manner as described in S144, and S256 is performed in the same manner as described in S146, and thus detail descriptions thereof will be omitted.

According to the above-described method, only the metal particles 120 may be detected, and it can be confirmed whether the biomolecules are or not present using the detected metal particles 120.

As described above, exemplary embodiments according to the present invention have been described, but are only exemplary, and it should be understood by those of ordinary skill in the art that the exemplary embodiments can be modified into various forms in the range of equivalents. Therefore, the exemplary embodiments of the present invention should be determined by the accompanying claims.

As described above, the present invention can effectively detect target biomolecules using metal particles and magnetic particles.

The method can actively detect target biomolecules and more rapidly detect the target biomolecules since the presence of the target biomolecules can be confirmed by detecting metal particles using an attractive force of an electric field.

The present invention can generate a non-uniform electric field using a first metal layer and a second metal layer having a fine irregular surface.

It should be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

1. A method of detecting biomolecules, comprising:
accommodating a plurality of metal particles to which a first molecule specifically binding to target biomolecules is attached, a plurality of magnetic particles to which a second molecule specifically binding to the target biomolecules is attached, and the biomolecules;
exerting an attractive force on the magnetic particles by applying a magnetic field;
filtering one or more among metal particles, biomolecules and metal particle-binding biomolecules, on which an attractive force is not exerted; and
exerting an attractive force on the metal particles of a first assembly in which both of the metal particles and the magnetic particles are bound to the biomolecules by applying an electric field and detecting the metal particles.

2. A method of detecting biomolecules, comprising:
accommodating a plurality of metal particles to which a first molecule specifically binding to target biomolecules is attached, a plurality of magnetic particles to which a second molecule specifically binding to the target biomolecules is attached, and the biomolecules;
exerting an attractive force on the magnetic particles by applying a magnetic field;
filtering one or more among metal particles, biomolecules and metal particle-binding biomolecules, on which an attractive force is not exerted;
cleaving one or more among specific bindings between the magnetic particles and the biomolecules of the first assembly in which both of the metal particles and the magnetic particles are bound to the biomolecules and specific bindings between the metal particles and the biomolecules of the first assembly; and
exerting an attractive force on the metal particles by applying an electric field and detecting the metal particles.

3. The method of claim 2, wherein the specific bindings are cleaved by applying ultrasonic waves.

4. The method of claim 1 or 2, wherein the filtering of the metal particles, the biomolecules and the metal particle-binding biomolecules comprises:
filtering the metal particles, the biomolecules and the metal particle-binding biomolecules, on which an attractive force is not exerted, by providing a solution.

5. The method of claim 1 or 2, further comprising:
when the magnetic particle is conductive, forming an insulating film on a surface of the magnetic particle.

6. The method of claim 1, wherein the exerting of an attractive force on the metal particles of a first assembly in which the metal particles, the biomolecules and the magnetic particles are bound by applying an electric field and detecting of the metal particles comprises:
generating an electric field by providing power to a first electrode and a second electrode of a metal particle-detecting structure including the first electrode and the second electrode, which are spaced apart from each other, a first metal layer formed on a surface of the first electrode and having a fine irregular surface and a second metal layer formed on a surface of the second electrode and having a fine irregular surface;
electrically connecting the first metal layer with the second metal layer when the metal particles of the first assembly are attached to one or more among the first metal layer and the second metal layer by the attractive force of the electric field or when the metal particles of the first assembly are present in a space between the first metal layer and the second metal layer; and
measuring electrical properties between the first metal layer and the second metal layer.

7. The method of claim 2, wherein the exerting of an attractive force on the metal particles by applying an electric field and detecting of the metal particles comprises:
generating an electric field by providing power to a first electrode and a second electrode of a metal particle-detecting structure including the first electrode and the second electrode, which are spaced apart from each other, a first metal layer formed on a surface of the first electrode and having a fine irregular surface and a second metal layer formed on a surface of the second electrode and having a fine irregular surface;
electrically connecting the first metal layer with the second metal layer when the metal particles are attached to one or more among the first metal layer and the second metal layer by the attractive force of the electric field or when the metal particles are present in the space between the first metal layer and the second metal layer; and
measuring electrical properties between the first metal layer and the second metal layer.

8. The method of claim 6 or 7, wherein the first electrode comprises:
a first body having a long rod shape; and
a plurality of first protrusions obliquely projecting from one side of the first body.

9. The method of claim 8, wherein the second electrode comprises:
a second body having a long rod shape; and
a plurality of second protrusions obliquely projecting from one side of the second body.

10. The method of claim 9, wherein the second protrusions are disposed in the space between the first protrusions.

11. The method of claim 6 or 7, wherein the electrical properties include one or more among variations of a resistance between the first metal layer and the second metal layer, a current flowing between the first metal layer and the second metal layer, and a voltage between the first metal layer and the second metal layer.

12. The method of claim 6 or 7, wherein the power provided to the first electrode and the second electrode is alternating current power.
